(19) United States
(12) Patent Application Publication
(10) Pub. No.: US 2010/0197915 A1
(43) Pub. Date: Aug. 5, 2010

(54) LAPATINIB INTERMEDIATES

(76) Inventors: Leonid METSGER, Beer-Sheva (IL); Slavik YURKOVSKI, Kiryat-Gat (IL); Sofia GOROHOVSKY-ROSENBERG, Beer Sheva (IL); Noa KIPNIS, Golan Heights (IL); Dikla LAVY, Beer Sheva (IL)

Correspondence Address: KENYON & KENYON LLP, ONE BROADWAY, NEW YORK, NY, 10004, US

(21) Appl. No.: 12/536,972

(22) Filed: Aug. 6, 2009

Related U.S. Application Data
(60) Provisional application No. 61/086,656, filed on Aug. 6, 2008.
(60) Provisional application No. 61/090,457, filed on Aug. 20, 2008.
(60) Provisional application No. 61/092,647, filed on Aug. 28, 2008.
(60) Provisional application No. 61/109,686, filed on Oct. 30, 2008.
(60) Provisional application No. 61/117,671, filed on Nov. 25, 2008.
(60) Provisional application No. 61/096,118, filed on Sep. 11, 2008.
(60) Provisional application No. 61/177,089, filed on May 11, 2009.

(51) Int. Cl. C07D 405/02; C07C 215/76

(52) U.S. Cl. 544/293; 564/443

(57) ABSTRACT

The invention provides lapatinib intermediates and improved processes for preparing lapatinib intermediates. The invention also provides processes for preparing lapatinib base and lapatinib ditosylate.

### RELATED APPLICATIONS

This application claims the benefit under 35 U.S.C. §119(e) of U.S. Provisional Application Nos. 61/086,656, filed on Aug. 6, 2008; 61/090,457, filed on Aug. 20, 2008; 61/092,647, filed on Aug. 28, 2008; 61/109,686, filed on Oct. 30, 2008; 61/117,671, filed on Nov. 25, 2008, 61/096,118, filed on Sep. 11, 2008; and 61/177,089, filed on May 11, 2009, each of which is incorporated herein by reference in its entirety.

### FIELD OF THE INVENTION

The invention relates to lapatinib intermediates and processes for preparing lapatinib intermediates. The invention also relates to processes for preparing lapatinib base and lapatinib ditosylate.

### BACKGROUND OF THE INVENTION

5-(4-[3-chloro-4-(3-fluorobenzyloxy)-anilino]-6-quinazolinyl)-furan-2-carbaldehyde monotosylate, compound D, having the following chemical structure:

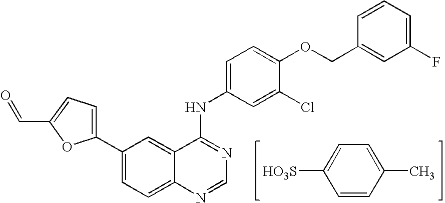

is known as lapatinib-aldehyde monotosylate.

5-(4-[3-chloro-4-(3-fluorobenzyloxy)-anilino]-6-quinazolinyl)-furan-2-carbaldehyde free base, the compound of formula D1, having the following chemical structure:

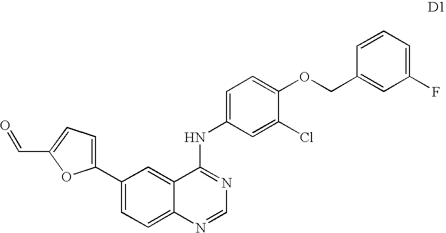

is known as lapatinib-aldehyde base.

Lapatinib-aldehyde monotosylate, the compound of formula D, lapatinib aldehyde ditosylate, and lapatinib-aldehyde base, the compound of formula D1, are intermediates in the preparation of lapatinib ditosylate, N-[3-chloro-4-[(3-fluorophenyl)methoxy]phenyl]-6-[5-[(2-methylsulfonylethylamino)methyl]-2-furyl]quinazolin-4-amine ditosylate, the compound of formula E, with the following chemical structure:

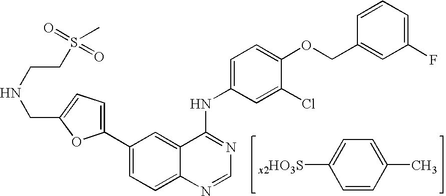

Lapatinib ditosylate is currently marketed in the United States under the tradename TYKERB® by GlaxoSmithKline. It was approved by the FDA as a drug for use in patients with advanced metastatic breast cancer.

Lapatinib ditosylate is described in PCT publications: WO 1999/035146, WO 2002/002552, WO 2005/046678, WO 2006/113649, WO 1998/002437, WO 2001/004111, WO 1996/009294, WO 2002/056912, WO 2005/105094, WO 2005/120504, WO 2005/120512, WO 2006/026313, and WO 2006/066267.

The above PCT publications describe the synthesis of lapatinib ditosylate as illustrated in the following Scheme:

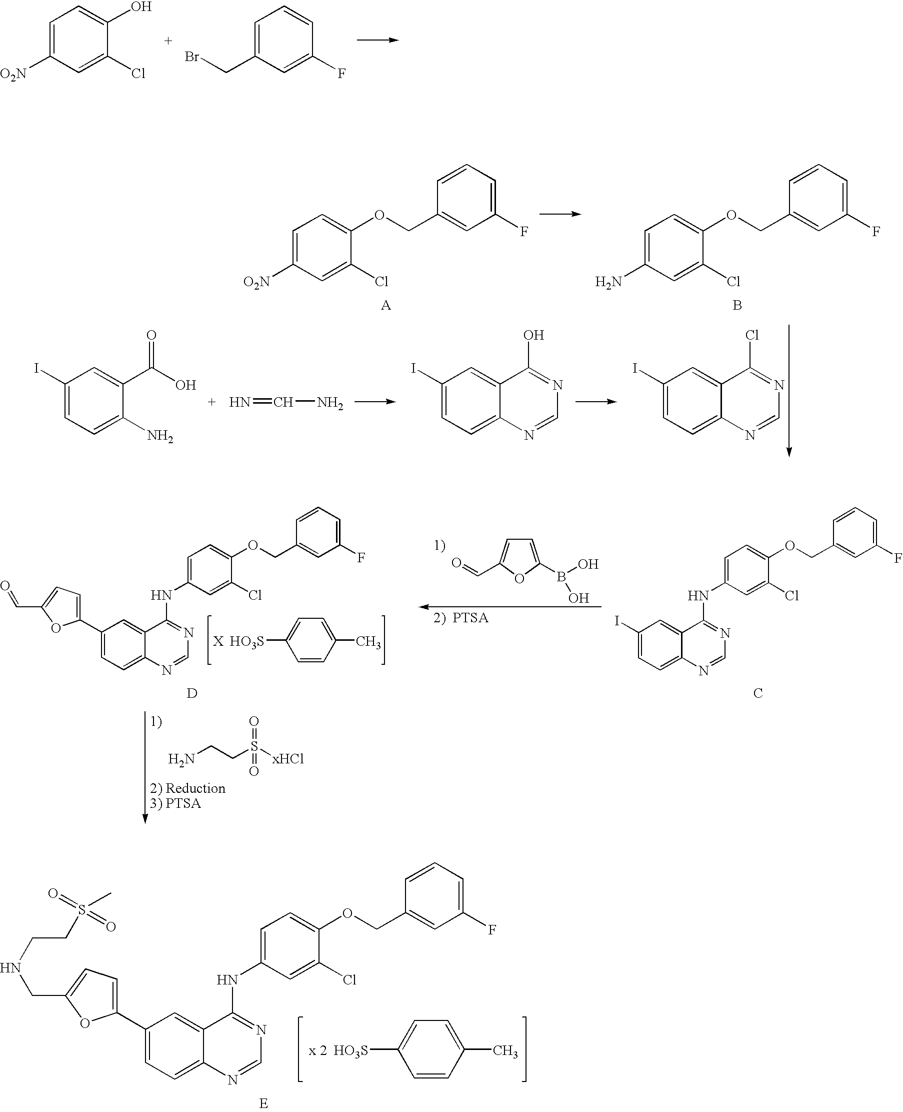

The processes described in PCT publications WO 98/02437, WO 99/35146, and WO 01/04111, encompass a reduction reaction to obtain compound of Formula B using Pt/C (5%) catalyst.

An embodiment of the present invention provides industrial applicable processes for preparing lapatinib aldehyde and salts thereof.

### SUMMARY OF THE INVENTION

An embodiment of the present invention encompasses lapatinib intermediates and improved processes for preparation and purification of lapatinib intermediates.

An embodiment of the present invention further provides for a lapatinib salt having purity levels of more than about 99.7%, more preferably, more than about 99.8%, and most preferably, more than about 99.9% area as determined by HPLC.

### DETAILED DESCRIPTION OF THE INVENTION

As used herein, the term volume (“V”) refers to ml per gram. For example, 30 V means 30 ml solvent per one gram of compound.

As used herein, the term “room temperature” (RT) refers to a temperature of about 20° C. to about 40° C.

As used herein, “des-chlorinated”, “des-fluorinated”, and “des-benzylated” by-products refer to compounds, which are formed as a result of the elimination of the chloro, fluoro or benzyl groups, or any combination thereof.

As used herein in connection with a measured quantity, the term “about” refers to that variation in the measured quantity as would be expected by the skilled artisan performing the measurement and exercising a level of care commensurate with the objective of the measurement and the precision of the measuring apparatus being used.

As used herein in connection with an isolated compound, the term “isolated” refers to a compound being isolated from a reaction mixture wherein the compound being isolated comprises at least 80% of the total composition.

In the present application, when the term “one-pot process” is used to describe the process for the preparation of the compound of Formula C from the compound of Formula A, it refers to a process in which the compound of Formula B is not separated from the reaction vessel. Therefore, processes involving separation and/or isolation of substances other than the compound of Formula B from one or more step of the processes are intended to be encompassed.

In one embodiment, the present invention encompasses 3-chloro-4-(3-fluorobenzyloxy)aniline, of the following Formula B:

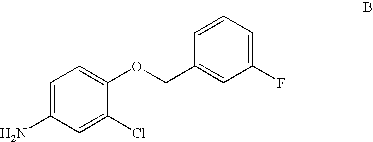

having less than 0.3% of any of 3-chloro-4-(benzyloxy)aniline, 4-(3-fluorobenzyloxy)aniline, 4-benzyloxyaniline, 3-chloro-4-hydroxyaniline, 4-aminophenol or combinations thereof, when measured as area by HPLC. In this application, unless specified otherwise, all HPLC purities are percent by area relative to the total area of the HPLC chromatogram (e.g., the total area of compound B and impurities).

Preferably, compound of Formula B contains less than 0.2%, more preferably, less than 0.1% of any of 3-chloro-4-(benzyloxy)aniline, 4-(3-fluorobenzyloxy)aniline, 4-benzyloxyaniline, 3-chloro-4-hydroxyaniline, and 4-aminophenol or combinations thereof, most preferably, isolated compound of Formula B has no trace amount, or at least undetectable amount by HPLC, of any of 3-chloro-4-(benzyloxy)aniline, 4-(3-fluorobenzyloxy)aniline, 4-benzyloxyaniline, 3-chloro-4-hydroxyaniline, and 4-aminophenol.

The present invention provides an improved process for preparing lapatinib intermediates having a low amount of des-chlorinated, des-benzylated, and des-fluorinated by-products.

The above mentioned compound of Formula B can be prepared according to a process comprising reducing a compound of Formula A, 3-chloro-4-(3-fluorobenzyloxy)nitrobenzene:

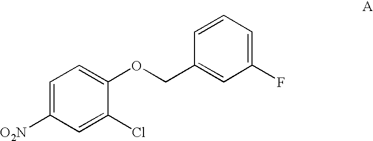

using an iron/ammonium chloride system, in the presence of a C₁-C₄ alcohol, and water. Preferably, the reaction mixture is heated to about reflux temperature for about 2 hours to about 30 hours, more preferably, about 6 hours to about 12 hours, even more preferably, about 4 hours to about 8 hours, and most preferably, about 2 hours. The compound of Formula B can be recovered from the reaction mixture by iron oxide filtration, main product extraction, and solvent evaporation. Preferably, the obtained compound of Formula B contains less than 0.3% of any of 3-chloro-4-(benzyloxy)aniline, 4-(3-fluorobenzyloxy)aniline, 4-benzyloxyaniline, 3-chloro-4-hydroxyaniline, 4-aminophenol or combinations thereof, when measured as area by HPLC.

Compound of Formula A can be prepared according to any process known in the art, for example, by the process disclosed in WO 98/02437, WO 99/35146, WO 01/04111, and WO 02/056912.

In another embodiment, the present invention encompasses a process for preparing lapatinib base or lapatinib ditosylate comprising preparing a compound of Formula B, which, when measured by HPLC, contains less than 0.3%, more preferably less than 0.2%, and even more preferably, less than 0.1% of any of 3-chloro-4-(benzyloxy)aniline, 4-(3-fluorobenzyloxy)aniline, 4-benzyloxyaniline, 3-chloro-4-hydroxyaniline, and 4-aminophenol or combinations thereof, as described above, and further converting the compound of Formula B to lapatinib base or lapatinib ditosylate.

In another embodiment, the present invention encompasses N-[3-chloro-4-(3-fluorobenzyloxy)-phenyl]-6-iodoquinazolin-4-amine, having the following Formula C:

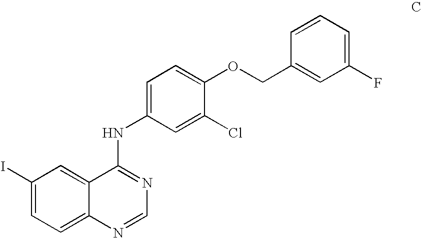

which, when measured by HPLC, has less than 0.3% of any of [3-chloro-4-(benzyloxy)-phenyl]-(6-iodoquinazolin-4-yl)-amine, [4-(3-fluorobenzyloxy)-phenyl]-(6-iodoquinazolin-4-yl)-amine, 4-benzyloxyphenyl-(6-iodoquinazolin-4-yl)-amine, 3-chloro-4-hydroxyphenyl-(6-iodoquinazolin-4-yl)-amine, 4-hydroxyphenyl-(6-iodoquinazolin-4-yl)-amine, or combination thereof.

Preferably, the compound of Formula C contains less than 0.2%, more preferably, less than 0.1%, for example, between 0.01% and 0.09%, of any of [3-chloro-4-(benzyloxy)-phenyl]-(6-iodoquinazolin-4-yl)-amine, [4-(3-fluorobenzyloxy)-phenyl]-(6-iodoquinazolin-4-yl)-amine, 4-benzyloxyphenyl-(6-iodoquinazolin-4-yl)-amine, 3-chloro-4-hydroxyphenyl-(6-iodoquinazolin-4-yl)-amine, 4-hydroxyphenyl-(6-iodoquinazolin-4-yl)-amine, or combination thereof. Most preferably, compound of Formula C contains no remains, or undetectable levels of any of [3-chloro-4-(benzyloxy)-phenyl]-(6-iodoquinazolin-4-yl)-amine, [4-(3-fluorobenzyloxy)-phenyl]-(6-iodoquinazolin-4-yl)-amine, 4-benzyloxyphenyl-(6-iodoquinazolin-4-yl)-amine, 3-chloro-4-hydroxyphenyl-(6-iodoquinazolin-4-yl)-amine, 4-hydroxyphenyl-(6-iodoquinazolin-4-yl)-amine, or combination thereof, when measured by HPLC.

In another embodiment, the present invention encompasses a one-pot process for the preparation of the compound of Formula C from the compound of Formula A. This process requires no handling of the genotoxic compound of Formula B.

The one-pot process for preparing a compound of Formula C comprises reducing a compound of Formula A:

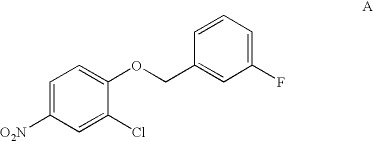

using an iron/ammonium chloride system, in the presence of a C₁-C₄ alcohol, and water; removing the iron oxide from the reaction mixture; removing the inorganic salt, preferably, by extraction with a mixture of water and dichloromethane; adding 4-chloro-6-iodoquinazoline and a second organic solvent to obtain a second reaction mixture; and heating the reaction mixture to obtain the compound of Formula C.

Preferably, the reaction mixture containing the compound of Formula A, the ammonium chloride, iron powder, the first organic solvent, and water, is heated to about reflux temperature, preferably, for about 2 hours to about 30 hours, more preferably, for about 6 hours to about 12 hours, more preferably for about 4 hours to about 8 hours, and most preferably, for about 2 hours.

The second organic solvent can be the same as the first organic solvent or can be selected from the group consisting of acetonitrile, dimethylsulfoxide, and C₁-C₄ alcohols. Preferably, the C₁-C₄ alcohol is ethanol or isopropanol. More preferably, the second organic solvent is isopropanol. Preferably, after the addition of the second organic solvent the reaction mixture is heated to a temperature of about reflux, for about 15 minutes to about 24 hours, more preferably about 30 minutes to about 12 hours. Preferably, the reaction mixture is heated for about 30 minutes to about an hour.

The compound of Formula C can be recovered from the reaction mixture by conventional methods, for example by trituration, filtration, extraction, and evaporation.

Preferably, the obtained compound of Formula C contains less than 0.3%, more preferably less than 0.2%, and even more preferably, less than 0.1% for example, between 0.01% and 0.09% of any of [3-chloro-4-(benzyloxy)-phenyl]-(6-iodoquinazolin-4-yl)-amine, [4-(3-fluorobenzyloxy)-phenyl]-(6-iodoquinazolin-4-yl)-amine, 4-benzyloxyphenyl-(6-iodoquinazolin-4-yl)-amine, 3-chloro-4-hydroxyphenyl-(6-iodoquinazolin-4-yl)-amine, 4-hydroxyphenyl-(6-iodoquinazolin-4-yl)-amine, or combination thereof, when measured by HPLC. Most preferably, the compound of Formula C contains no remaining or undetectable levels of any of [3-chloro-4-(benzyloxy)-phenyl]-(6-iodoquinazolin-4-yl)-amine, [4-(3-fluorobenzyloxy)-phenyl]-(6-iodoquinazolin-4-yl)-amine, 4-benzyloxyphenyl-(6-iodoquinazolin-4-yl)-amine, 3-chloro-4-hydroxyphenyl-(6-iodoquinazolin-4-yl)-amine, 4-hydroxyphenyl-(6-iodoquinazolin-4-yl)-amine, or combination thereof, when measured by HPLC.

In another embodiment, the present invention encompasses a process for the purification of compound of Formula C, comprising recovering compound of Formula C form a mixture of compound C and a C₁-C₄ alcohol, preferably, methanol. Preferably, the amount of the C₁-C₄ alcohol is about 10V to about 40V, more preferably about 20V.

In another embodiment, the present invention encompasses a process for preparing lapatinib base or lapatinib ditosylate, comprising preparing a compound of Formula C, having less than 0.3%, more preferably, less than 0.2%, and even more preferably, less than 0.1%, for example, between 0.01% and 0.09% of any of [3-chloro-4-(benzyloxy)-phenyl]-(6-iodoquinazolin-4-yl)-amine, [4-(3-fluorobenzyloxy)-phenyl]-(6-iodoquinazolin-4-yl)-amine, 4-benzyloxyphenyl-(6-iodoquinazolin-4-yl)-amine, 3-chloro-4-hydroxyphenyl-(6-iodoquinazolin-4-yl)-amine, 4-hydroxyphenyl-(6-iodoquinazolin-4-yl)-amine, or combination thereof, when measured by HPLC, as described above, and further converting it to lapatinib base or lapatinib ditosylate.

In another embodiment, the present invention encompasses 5-{4-[3-chloro-4-(3-fluorobenzyloxyl)-phenylamino]-quinazolin-6-yl}-furan-2-carbaldehyde monotosylate, compound of Formula D (denominated as lapatinib aldehyde monotosylate),

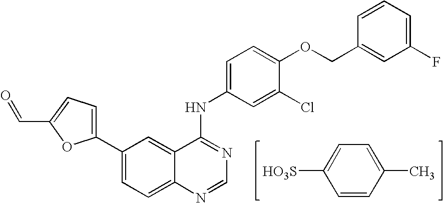

having less than 0.3% of any of 5-[4-[3-chloro-4-(benzyloxy)-phenylamino]-quinazolin-6-yl]-furan-2-carbalehyde, 5-[4-[4-(3-fluorobenzyloxy)-phenylamino]-quinazolin-6-yl]-furan-2-carbalehyde, 5-[4-(4-benzyloxyphenylamino)-quinazolin-6-yl]-furan-2-carbalehyde, 5-[4-(3-chloro-4-hydroxyphenylamino)-quinazolin-6-yl]-furan-2-carbalehyde, and 5-[4-(4-hydroxyphenylamino)-quinazolin-6-yl]-furan-2-carbalehyde or combination thereof, when measured by HPLC.

Preferably, the compound of Formula D contains less than 0.2%, more preferably, less than 0.1%, for example, between 0.01% and 0.09% of any of 5-[4-[3-chloro-4-(benzyloxy)-phenylamino]-quinazolin-6-yl]-furan-2-carbaldehyde, 5-[4-[4-(3-fluorobenzyloxy)-phenylamino]-quinazolin-6-yl]-furan-2-carbaldehyde, 5-[4-(4-benzyloxyphenylamino)-quinazolin-6-yl]-furan-2-carbaldehyde, 5-[4-(3-chloro-4-hydroxyphenylamino)-quinazolin-6-yl]-furan-2-carbaldehyde, and 5-[4-(4-hydroxyphenylamino)-quinazolin-6-yl]-furan-2-carbaldehyde or combinations thereof. Most preferably, compound D contains no remaining, or undetectable levels of any of 5-[4-[3-chloro-4-(benzyloxy)-phenylamino]-quinazolin-6-yl]-furan-2-carbaldehyde, 5-[4-[4-(3-fluorobenzyloxy)-phenylamino]-quinazolin-6-yl]-furan-2-carbaldehyde, 5-[4-(4-benzyloxyphenylamino)-quinazolin-6-yl]-furan-2-carbaldehyde, 5-[4-(3-chloro-4-hydroxyphenylamino)-quinazolin-6-yl]-furan-2-carbaldehyde, and 5-[4-(4-hydroxyphenylamino)-quinazolin-6-yl]-furan-2-carbaldehyde, when measured by HPLC.

In another embodiment, the present invention encompasses isolated 5-{4-[3-chloro-4-(3-fluorobenzyloxyl)-phenylamino]-quinazolin-6-yl}-furan-2-carbaldehyde, compound of Formula D1 (denominated as lapatinib aldehyde base). Preferably, compound D1 is solid.

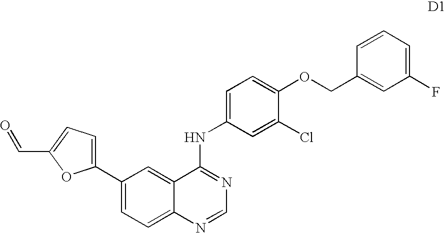

Preferably, compound D1 contains less than 0.3% of any of 5-[4-[3-chloro-4-(benzyloxy)-phenylamino]-quinazolin-6-yl]-furan-2-carbaldehyde, 5-[4-[4-(3-fluorobenzyloxy)-phenylamino]-quinazolin-6-yl]-furan-2-carbaldehyde, 5-[4-(4-benzyloxyphenylamino)-quinazolin-6-yl]-furan-2-carbaldehyde, 5-[4-(3-chloro-4-hydroxyphenylamino)-quinazolin-6-yl]-furan-2-carbaldehyde, and 5-[4-(4-hydroxyphenylamino)-quinazolin-6-yl]-furan-2-carbaldehyde or combination thereof, when measured by HPLC.

Preferably, the compound of Formula D1 contains less than 0.2%, more preferably, less than 0.1%, for example, between 0.01% and 0.09% of any of 5-[4-[3-chloro-4-(benzyloxy)-phenylamino]-quinazolin-6-yl]-furan-2-carbaldehyde, 5-[4-[4-(3-fluorobenzyloxy)-phenylamino]-quinazolin-6-yl]-furan-2-carbaldehyde, 5-[4-(4-benzyloxyphenylamino)-quinazolin-6-yl]-furan-2-carbaldehyde, 5-[4-(3-chloro-4-hydroxyphenylamino)-quinazolin-6-yl]-furan-2-carbaldehyde, and 5-[4-(4-hydroxyphenylamino)-quinazolin-6-yl]-furan-2-carbaldehyde or combinations thereof. Most preferably, compound D1 contains no remaining, or undetectable levels of any of 5-[4-[3-chloro-4-(benzyloxy)-phenylamino]-quinazolin-6-yl]-furan-2-carbaldehyde, 5-[4-[4-(3-fluorobenzyloxy)-phenylamino]-quinazolin-6-yl]-furan-2-carbaldehyde, 5-[4-(4-benzyloxyphenylamino)-quinazolin-6-yl]-furan-2-carbaldehyde, 5-[4-(3-chloro-4-hydroxyphenylamino)-quinazolin-6-yl]-furan-2-carbaldehyde, and 5-[4-(4-hydroxyphenylamino)-quinazolin-6-yl]-furan-2-carbaldehyde, when measured by HPLC.

Another embodiment of the present invention provides an improved process for preparing lapatinib ditosylate. This improved route of synthesis is carried out via the lapatinib aldehyde base instead of the lapatinib aldehyde monotosylate intermediate. The inventors have discovered that during the preparation of lapatinib aldehyde monotosylate, the alkyl esters of the p-toluenesulfonic acid are formed, not to be limited by any mechanism, due to a reaction between the p-toluenesulfonic acid and the alcohol, which serves as the solvent of the reaction. By avoiding the preparation of lapatinib aldehyde monotosylate, the formation of these genotoxic esters is prevented as well.

In one embodiment, the present invention encompasses a process for preparing lapatinib aldehyde base comprising heating a reaction mixture of a compound of Formula C, 5-formyl-2-furanboronic acid, a palladium catalyst selected from the group consisting of palladium(II) acetate, palladium(II) chloride, and palladium tetrakistriphenylphosphine, a base, and a polar organic solvent, preferably, a mixture of ethanol and tetrahydrofuran; removing the salts from the reaction mixture; and recovering the lapatinib aldehyde base as precipitate from the reaction mixture.

Preferably, the reaction mixture is heated to about 40° C. to about 110° C., more preferably, about 60° C. to about 110° C., more preferably, about 60° C. to about 90° C., most preferably about 78° C. Preferably, the reaction mixture is heated for about 1 hour to about 24 hours, more preferably, for about 1.5 hours to about 6 hours.

Optionally, the obtained lapatinib aldehyde base is further combined with p-toluenesulfonic acid (“PTSA”), preferably with about 1 equivalent to about 2 equivalents of p-toluenesulfonic acid, to obtain lapatinib aldehyde monotosylate.

The palladium catalyst can be selected from the group consisting of palladium(II) acetate, palladium(II) chloride, and palladium tetrakistriphenylphosphine. The most preferred palladium catalyst is palladium(II) acetate.

Suitable bases include, but are not limited to, alkali carbonates, alkali bicarbonates, alkali acetates, alkali phosphates, alkali hydroxide, aliphatic tertiary amines or diamines, wherein the alkyl groups are independently selected from the group consisting of C₁-C₄ alkyls, C₂-C₁₀ cyclic or polycyclic tertiary amines or diamines, or tertiary amines or diamines consisted from any C₁-C₄ aliphatic or aromatic or heterocylic group. Preferably the base is potassium carbonate or diisopropylethylamine, and most preferably the base is diisopropylethylamine. The polar organic solvent can be selected from the group consisting of tetrahydrofuran, dioxane, C₁-C₄ alcohols, C₂-C₁₀ glycols, glycerol, and mixtures thereof. Preferably, the organic solvent is ethanol, more preferably, the polar organic solvent is a mixture of ethanol and tetrahydrofuran. Preferably the mixture contains between about 10% to about 75% tetrahydrofuran, more preferably between about 10% to about 60% tetrahydrofuran, and most preferably between about 10% to about 40% by volume.

The salts can be removed from the reaction mixture by any conventional method, such as by filtration.

Lapatinib aldehyde base can then be recovered from the reaction mixture using any method known in the art, for example, the reaction mixture may be cooled to induce precipitation at a temperature of about −5° C. to about 25° C., more preferably to a temperature of about 0° C. to about 20° C., and most preferably to a temperature of about 0° C. to about 10° C. Optionally, the lapatinib aldehyde base is seeded using a small amount of the pure product. The reaction mixture can then be maintained at the same final temperature for at least an hour to increase precipitation. The obtained lapatinib aldehyde base can be further dried; preferably drying is carried out under about 1 atmosphere, more preferably, less than about 100 mmHg, preferably at a temperature of about 25° C. to about 40° C.

Optionally, after removing the salts by filtration, the filtrate is maintained at a temperature of about 5° C. to about 65° C., more preferably at a temperature of about 20° C. to about 60° C., and most preferably at a temperature of about 20° C. to about 40° C.; and the PTSA is added drop-wise as an aqueous solution. Preferably, about 1 equivalent to about 2 equivalents of p-toluenesulfonic acid are added. The reaction mixture can be maintained at the same temperature for about at least an hour.

Lapatinib aldehyde monotosylate can be recovered from the reaction mixture using any method known in the art, for example, the reaction mixture may be cooled to a temperature of about 0° C. to about 25° C. to induce precipitation, and the lapatinib aldehyde monotosylate can be collected by filtration. The obtained product can be further dried under reduced pressure preferably under about 1 atmosphere, preferably, less than about 100 mmHg, preferably at a temperature of about 20° C. to about 50° C., more preferably about 25° C. to about 40° C. Preferably, the precipitate is dried for about 6 hours to about 30 hours, more preferably, about 12 hours to about 24 hours.

Preferably, the obtained compound of Formula D or D1 contains less than 0.3%, more preferably, less than 0.2%, and more preferably, less than 0.1% of any of 5-[4-[3-chloro-4-(benzyloxy)-phenylamino]-quinazolin-6-yl]-furan-2-carbaldehyde, 5-[4-[4-(3-fluorobenzyloxy)-phenylamino]-quinazolin-6-yl]-furan-2-carbaldehyde, 5-[4-(4-benzyloxyphenylamino)-quinazolin-6-yl]-furan-2-carbaldehyde, 5-[4-(3-chloro-4-hydroxyphenylamino)-quinazolin-6-yl]-furan-2-carbaldehyde, and 5-[4-(4-hydroxyphenylamino)-quinazolin-6-yl]-furan-2-carbaldehyde or combination thereof, when measured by HPLC.

In another embodiment, the present invention encompasses a process for purifying lapatinib aldehyde monotosylate comprising trituration with an organic solvent selected from the group consisting of C₁-C₄ alcohols, acetone, acetonitrile and tetrahydrofuran. Preferably, the organic solvent used is methanol, ethanol, isopropanol, or acetone, most preferably the organic solvent is methanol.

Preferably, triturating is performed at a temperature of about 20° C. to about 90° C., and more preferably, about 40° C. to about 60° C., preferably for about an hour to about 12 hours. Preferably, about 5V to about 20V of solvent are applied, more preferably about 10V.

In another embodiment, the present invention encompasses a process for preparing compound D1 comprising converting compound D to compound D1 in the presence of an inorganic base.

The inorganic base can be selected from the group consisting of alkali carbonates, alkali bicarbonates, alkali acetates, alkali phosphates, and alkali hydroxide. The most preferred inorganic base is potassium carbonate. The reaction may be carried out in the presence of a solvent selected from the group consisting of acetonitrile, acetone, water, ethyl acetate, dichloromethane, and tetrahydrofuran.

In another embodiment, the present invention encompasses a process for preparing lapatinib base comprising providing a first reaction mixture containing lapatinib aldehyde base or its salt, methylsulfonylethylamine or its hydrochloride salt, acetic acid, an inorganic base, and an organic solvent; adding a reducing agent to form a second reaction mixture; adding water and separating the organic phase from the aqueous phase; and recovering the precipitated lapatinib base from the organic phase.

The use of the free base of lapatinib aldehyde, provided in this invention, has an advantage as compared to the use of lapatinib aldehyde monotosylate, as it prevents the formation of genotoxic alkyl tosylate ester. Additionally, it can reduce the amount of additional salt formed after the basification of the reaction mixture.

Suitable inorganic bases can be, but are not limited to, alkali carbonates, alkali bicarbonates, alkali phosphates, and alkali acetates, most preferably, sodium acetate or potassium acetate. Preferably, the reducing agent is sodium triacetoxyborohydride.

This new synthesis avoids the use of an organic base, such as N,N-diisopropylethylamine, that possesses high solubility in organic solvents, and therefore may interfere with the isolation of the lapatinib base.

The organic solvent can be selected from the group consisting of dimethylformamide, dimethylacetamide, dichloromethane, dimethyl carbonate, diethyl carbonate, toluene, acetonitrile, tetrahydrofuran, and C₁-C₆ esters, more preferably, C₁-C₄ esters, most preferably, ethyl acetate, methyl acetate, and isobutyl acetate. When toluene, acetonitrile, or tetrahydrofuran is used as the organic solvent, the second reaction mixture is evaporated to dryness, before the extraction step, and the obtained residue is then dissolved in a mixture of water and ethyl acetate or a mixture of water and isobutyl acetate. Most preferably, the organic solvent is selected from the group consisting of ethyl acetate, tetrahydrofuran, dimethylformamide, and dimethylacetamide.

Preferably, the first reaction mixture is maintained at about room temperature for about 15 minutes to about 24 hours, preferably about 30 minutes to about 3 hours, more preferably about an hour. Preferably, the second reaction mixture is maintained at about 0° C. to about room temperature, more preferably about room temperature for about an hour to about 24 hours, more preferably for about 1.5 hours to about 2.5 hours.

Preferably, before separating the organic phase from the aqueous phase, additional aqueous solution of an inorganic base is added to the second reaction mixture. The inorganic base can be selected from the group consisting of alkali carbonates, alkali bicarbonates, and alkali hydroxide, most preferably, sodium hydroxide, or sodium carbonate.

The obtained lapatinib base can be extracted from the reaction mixture by any conventional technique. For example, extraction can be achieved by evaporation, and re-crystallization.

In another embodiment, the present invention encompasses a process for preparing lapatinib base comprising providing a first reaction mixture containing compound D1 or its salt, methylsulfonylethylamine or its hydrochloride salt, acetic acid, N,N-diisopropylethylamine, ethyl acetate, and/or tetrahydrofuran, and a water miscible organic solvent such as dimethylformamide, and dimethylacetamide; adding a reducing agent to form a second reaction mixture; adding water and sodium hydroxide, and separating the organic phase from the aqueous phase; and extracting the lapatinib base from the organic phase.

Preferably, the reducing agent is sodium triacetoxyborohydride.

Preferably, the first reaction mixture is maintained at about room temperature for about 15 minutes to about 24 hours, preferably about 30 minutes to about 3 hours, more preferably about an hour. Preferably, the second reaction mixture is maintained at about 0° C. to about room temperature, more preferably about room temperature for about an hour to about 24 hours, more preferably for about 1.5 hours to about 2.5 hours.

Preferably, before separating the organic phase from the aqueous phase, an inorganic base is added to the second reaction mixture. The inorganic base can be selected from the group consisting of alkali carbonates, alkali bicarbonates, and alkali hydroxide, most preferably, sodium hydroxide, or sodium carbonate.

In another embodiment, the present invention encompasses a process for purifying lapatinib base comprising providing a suspension or a solution of lapatinib base and an organic solvent selected from the group consisting of C₃-C₇ ketones (i.e., ketones having a total of 3 to 7 carbons), preferably acetone, acetonitrile, ethyl acetate, methyl acetate, isobutyl acetate, and dichloromethane; and collecting the precipitate. Most preferably, the organic solvent is ethyl acetate. Preferably, the amount of the solvent is about 4V to about 30V, more preferably about 4V to about 20V, and most preferably, about 4V to about 10V.

The obtained lapatinib base is preferably obtained in a total purity level of more than 98%, weight percentage as measured by HPLC. More preferably the lapatinib base is obtained in a total purity level of more than 99% and most preferably, more than 99.5%.

Those skilled in the art would understand, that repeating the processes described above, would provide lapatinib base in a higher degree of purity.

The suspension can be maintained at about 0° C. to about room temperature, preferably at about room temperature before filtering the precipitate.

In another embodiment, the present invention encompasses a process for preparing lapatinib ditosylate, or any other lapatinib salt, comprising obtaining lapatinib base and further converting it to lapatinib salt, preferably, lapatinib ditosylate.

In another embodiment, the present invention encompasses lapatinib salt having purity levels of more than about 99.7%, more preferably, more than about 99.8%, and most preferably, more than about 99.9%, for example between 99.7% and 99.999%, weight percentage as measured by HPLC.

Preferably, the lapatinib salt of the present invention contains undetectable levels of any des-benzylated, des-chlorinated, and des-fluorinated by-products of lapatinib, when measured by HPLC.

Preferably, the lapatinib salt, contain less than 0.2%, and more preferably, less than 0.1%, for example, between 0.01% and 0.09% of des-benzylated, des-chlorinated, and des-fluorinated derivatives derived from lapatinib.

The lapatinib salt can be a salt of an acid selected from the group consisting of hydrochloric acid, hydrobromide acid, phosphoric acid, sulfuric acid, methane sulphonic acid, mono organic acids, and diorganic acid. Suitable mono-organic acids are, but are not limited to, acetic acid, or formic acid. Suitable diorganic acids include, but are not limited to, tartaric acid, succinic acid, p-toluenesulfonic acid, and maleic acid.

The present invention further encompasses a modified process for preparing lapatinib ditosylate, comprising the reductive amination of compound of Formula D or compound of Formula D1 without the use of sodium triacetoxyborohydride, which often leads to a production of high amounts of inorganic borates and acetates and higher costs.

In another embodiment, the present invention encompasses a one pot process for preparing lapatinib ditosylate, comprising: combining compound of Formula D, or compound of Formula D1, and methylsulfonylethylamine free base or its salt, in the presence of an organic solvent, an organic base, acetic acid, and a reducing agent to obtain lapatinib base; forming a mixture of about 15V to about 30V of ethyl acetate, about 3V to about 7V of tetrahydrofuran, and about 1V to about 10V of dimethylformamide, and water; separating the organic solution from the aqueous solution; and adding p-toluenesulfonic acid to obtain lapatinib ditosylate. Optionally, dimethylacetamide is used instead of dimethylformamide.

Preferably, the organic solvent is selected from the group consisting of ethyl acetate, dichloromethane, N,N-dimethylformamide, N,N-dimethylacetamide, acetonitrile, tetrahydrofuran, dimethoxyethane, methyl tert butyl ether, toluene, and mixtures thereof. Preferably, the organic base is a trialkylamine, wherein the alkyl groups are independently selected from the group consisting of C₁-C₄ alkyls. Preferably, the organic base is diisopropylamine. Preferably, the reducing agent is selected from the group consisting of alkali borohydrides or alkali cyanoborohydrides. Preferably, the reducing agent is selected from the group consisting of sodium triacetoxyborohydride, sodium borohydride, and sodium cyanoborohydride; more preferably, sodium borohydride, and sodium triacetoxyborohydride, and most preferably, sodium triacetoxyborohydride.

Typically, the mixture of compound of Formula D, or compound of Formula D1, with methylsulfonylethylamine free base or its salt, the organic solvent, the organic base, and acetic acid is maintained at a temperature of about 0° C. to about 30° C. for about an hour; preferably, the mixture is maintained at a temperature of about 10° C. to about 20° C. The reducing agent may be then added in portion within about 30 minutes, and the mixture is further maintained at a temperature of about 0° C. to about 30° C.; preferably, at about 10° C. to about 20° C.; for about an hour to about 24 hours.

In another embodiment, the present invention encompasses a process for the purification of lapatinib ditosylate, comprising triturating from methanol.

In another embodiment, the present invention encompasses a process for preparing lapatinib ditosylate, comprising the following steps:

a) reducing a compound of Formula A using an iron/ammonium chloride system, in the presence of a C₁-C₄ alcohol, and water; removing the iron oxide from the reaction mixture; removing the inorganic salt, preferably, by extracting from a mixture of water and dichloromethane; adding 4-chloro-6-iodoquinazoline and a second organic solvent to obtain a second reaction mixture; and heating the reaction mixture to obtain the compound of Formula C;
b) heating a reaction mixture of a compound of Formula C, 5-formyl-2-furanboronic acid, a palladium catalyst selected from the group consisting of palladium(II) acetate, palladium(II) chloride, and palladium tetrakistriphenylphosphine, a base, and a polar organic solvent, preferably, a mixture of ethanol and tetrahydrofuran; removing the salts from the reaction mixture; and recovering lapatinib aldehyde base, Formula D1, as precipitate from the reaction mixture; optionally, p-toluenesulfonic acid is added to obtain lapatinib aldehyde monotosylate, Formula D; and
c) combining compound of Formula D, or compound of Formula D1, and methylsulfonylethylamine free base or its salt, in the presence of an organic solvent, an organic base, and acetic acid; adding a reducing agent to obtain lapatinib base; forming a mixture of about 15V to about 30V of ethyl acetate, about 3V to about 7V of tetrahydrofuran, and about 1V to about 10V of dimethylformamide, and water; separating the organic solution from the aqueous solution; and adding p-toluenesulfonic acid to obtain lapatinib ditosylate. Optionally, dimethylacetamide is used instead of dimethylformamide.

In another embodiment, the present invention encompasses lapatinib base or salt made by the processes described above. Preferably, the lapatinib salt is lapatinib ditosylate.

### EXAMPLES

### HPLC Method for Measuring Chemical Purity (For B and C)

| Column: | Zorbax SB C8 100 × 4.6 mm 1.8 μm |
|---|---|
| Mobile phase: | (A) 80% (0.02M KH₂PO₄ pH 5.0): 20% ACN |
| | (B) ACN |
| Gradient: | From 0 to 2 min 80% (A): 20% (B) isocratically |
| | From 2 to 22 min (B) increases from 20 to 75% |
| | From 22 to 30 min 25% (A): 75% (B) isocratically |
| Detection: | 210 nm |
| Flow: | 1 mL/min |
| Detection limit: | 0.03% |

### HPLC Method for Measuring Chemical Purity (For D, D1, and E):

| Column: | Zorbax SB-Phenyl 100 × 4.6 mm 1.8 μm |
|---|---|
| Mobile phase: | (A) 80% (0.02M KH₂PO₄ pH 5.0): 20% ACN |
| | (B) ACN |
| Gradient: | From 0 to 18 min 70% (A): 30% (B) isocratically |
| | From 18 to 32 min (B) increases from 30 to 65% |
| | From 32 to 40 min 35% (A): 65% (B) isocratically |
| Detection: | 210 nm |
| Flow: | 1.5 mL/min |
| Detection limit: | 0.03% |

### Example 1

### Preparation of Lapatinib Aldehyde Monotosylate

In a 500 ml round-bottomed flask were added 10 g of compound of Formula C, 3.7 g of 5-formyl-2-furanboronic acid, 0.2 g of palladium(II) acetate, 5.5 g of Potassium carbonate and 150 ml of absolute ethanol. The suspension was stirred and heated to reflux for 30 minutes. The reaction mixture was cooled to 25° C. and diluted with 150 ml of THF. Inorganic salts were filtered off in vacuum and discarded. The filtrate was transferred into 500 ml round-bottomed flask and heated to 60° C. The solution of 11.3 gr of p-toluenesulfonic acid in 15 ml of water was added drop-wise. The resulting light-orange suspension was stirred at 60° C. for an 1 hour. Then, the heating source was removed and the mixture was stirred at 25° C. for 2 hours, cooled to 5° C. and stirred for 0.5 hours. The precipitated yellow solid was filtered in vacuum and washed over the filter with absolute ethanol (3×50 ml). It was allowed to dry in a vacuum oven at 25° C. for 16 hours to give 15.7 g of the final product, which was identified as lapatinib aldehyde monotosylate. Purity 97.95%

### Example 2

### Purification of Lapatinib Aldehyde Monotosylate

2 g of LPT (lapatinib) aldehyde monotosylate (Purity by HPLC: 95.2%) and 20 ml of methanol were added to a 100 ml round-bottomed flask. The resulting suspension was stirred for 2.5 h at 35-40° C. The solid was vacuum-filtered and washed with 5 ml of fresh methanol. The resulting cake was dried in a vacuum oven at 40° C. for 16 hours to obtain lapatinib aldehyde monotosylate (1.50 gr, yield—75%). Purity 97.8%

### Example 3

### Purification of Lapatinib Aldehyde Monotosylate

2 g of lapatinib aldehyde monotosylate and 20 ml of ethanol were added to a 100 ml round-bottomed flask equipped with a magnetic stirrer. The resulting suspension was stirred for 2.5 hours at 35-40° C. The solid was vacuum-filtered and washed with 5 ml of fresh solvent ethanol. The resulting cake was dried in a vacuum oven at 40° C. for 16 hours to obtain lapatinib aldehyde monotosylate (1.74 gr, yield—87%).

### Example 4

### Preparation of Lapatinib Aldehyde Monotosylate

In a 100 ml round-bottomed flask were added 1 g of compound of Formula C, 0.37 g of 5-formyl-2-furanboronic acid, 0.02 g of palladium(II) acetate, 0.55 gr of Potassium carbonate and 15 ml of absolute ethanol. The suspension was stirred and heated to reflux for 0.5 hours. The reaction mixture was cooled to RT and diluted with 15 ml of THF. Inorganic salts were filtered off in vacuum and discarded. The filtrate was transferred into 100 ml round-bottomed flask and heated to 60-65° C. The solution of 1.5 gr of p-toluenesulfonic acid in 2 ml of water was added dropwise during 5 min. The resulting light-orange suspension was stirred at 60-65° C. for an hour. Then, the heating source was removed and the mixture was stirred at 25° C. for 2 hours, cooled to 5° C. and stirred for 0.5 hours. The precipitated orange solid was filtered in vacuum and washed over the filter with absolute ethanol (2×2.5 ml). The resulting cake was allowed to dry in a vacuum oven at 25° C. for 16 hours to give 1.4 g of the final product, which was identified as lapatinib aldehyde monotosylate. Purity by HPLC: 89.66%

### Example 5

### Preparation of 3-Chloro-4-(3-fluorobenzyloxy)aniline

35.6 g of “Intermediate-A” (compound of Formula A), 21.2 g of iron powder (70 mesh), 60.9 g of ammonium chloride, 506 ml of ethanol and 128 ml of water were refluxed for 2 hours in 1 L reactor. The reaction mixture was cooled to 20-25° C. and separated from insoluble iron oxide by vacuum filtration. The filtered solids were washed with ethanol. The resulting filtrate was evaporated to obtain 94.9 g of highly wet orange solid. All the solid mass was mixed with 400 ml of dichloromethane to dissolve organic product. The resulting turbid solution was filtered to remove inorganic salts. The filtrate was charged into separation funnel to remove aqueous residue. The organic phase was dried over anhydrous sodium sulfate and evaporated to dryness to obtain 30.8 g (Yield: 96%) of the 3-chloro-4-(3-fluorobenzyloxy)aniline of Formula B (purity determined by HPLC: 99.67%, most significant impurity: 0.10%).

### Example 6

### N-[3-Chloro-4-(3-Fluorobenzyloxy)-Phenyl]-6-Iodoquinazolin-4-amine preparation (one-pot process)

30.0 g of “Intermediate-A” (compound of Formula A), 17.8 g of iron powder (70 mesh), 51.3 g of ammonium chloride, 432 ml of ethanol and 108 ml of water were refluxed for 5 hours in 1 L reactor equipped with mechanical stirrer and condenser. The reaction mixture was then cooled to 20-25° C. and separated from insoluble iron oxide by vacuum filtration. The filtered solids were washed with ethanol (4×100 ml). The resulting filtrate was evaporated from reactor under reduced pressure to resulting in a wet orange residue. The residue was dissolved in 350 ml of dichloromethane and 300 ml water. The separated organic phase was washed with water (2×300 ml). The obtained organic solution was concentrated to about 150 ml followed by addition of 300 ml iso-propanol. The mixture was concentrated to 300 ml followed by addition 150 ml iso-propanol. The resulting mixture was concentrated to 300 ml. Then iso-propanol was added to obtain a final volume of about 570 ml (purity determined by HPLC: 98.5%).

23.8 g of 4-chloro-6-iodoquinazoline were added to the organic solution, heated to reflux, stirred for 30 minutes and then cooled to 20-25° C. The slurry was filtered and washed with 110 ml iso-propanol to obtain 53.5 g of wet crude product. Then it was triturated in 830 ml of boiling acetone for an hour, cooled and filtered. The product was triturated twice again each time in 655 ml of boiling acetone for an hour, cooled and filtered. Finally it was dried at 25° C. in vacuum oven to afford 32.2 g of “Intermediate-C” (Yield: 60%, Purity: 92.62%).

### Example 7

### N-[3-Chloro-4-(3-Fluorobenzyloxy)-Phenyl]-6-Iodoquinazolin-4-amine purification

2.0 g of “Intermediate-C” (compound of Formula C), which contained 6.7% of 4-hydroxy-6-iodoquinazoline (as determined by HPLC) was combined with 40 ml methanol and refluxed. The hot turbid solution was then filtered to obtain a filtrate, which was cooled to 20-25° C. The precipitated material was filtered to obtain 1.60 gr (yield—80%) of pure N-[3-Chloro-4-(3-Fluorobenzyloxy)-Phenyl]-6-Iodoquinazolin-4-amine (purity determined by HPLC: 99.5%).

### Example 8

### N-[3-Chloro-4-[(3-fluorobenzyl)oxy]phenyl]-6-[2-formylfuryl]-4-quinazolinamine monotosylate purification

2.0 g of “Intermediate-D” (compound of Formula D) (purity determined by HPLC—95.2%) was triturated in 20 ml of iso-propanol at 40° C. for 2.5 hours, filtered, washed with the same solvent and dried to afford 1.91 g (yield—95%) of pure material (purity by determined HPLC: 99.0%).

### Example 9

### N-[3-Chloro-4-[(3-fluorobenzyl)oxy]phenyl]-6-[5-[[[2-(methylsulfonyl)ethyl]amino]methyl]-2-furyl]-4-quinazolinamine ditosylate (lapatinib Ditosylate) purification

1.0 g of lapatinib Ditosylate (purity determined by HPLC: 98.0%) was triturated in 10 ml of methanol at 40° C. for 2.5 hours, filtered, washed with the same solvent and dried to afford 0.92 g (yield—92%) of pure material (purity determined by HPLC: 98.5%).

### Example 10

2.0 gr “Intermediate-D” (compound of Formula D), 0.8 gr methylsulfonylethylamine hydrochloride, 20 ml THF, 0.71 ml acetic acid and 2.15 ml N,N-diisopropylethylamine were mixed at 25-30° C. The resulting reaction mixture was stirred at 25-30° C. for an hour. Afterwards, 0.24 gr of sodium borohydride were added in two equal portions with 30 min interval (2×0.12 gr). The resulting mixture was stirred at 25-30° C. for an additional hour. The reaction mixture was quenched with 6 ml of 25% NaOH aq. and 4 ml of water. The resulting mixture was stirred for 0.5 hours. The aqueous phase was separated and washed with 5 ml of THF. The combined THF extracts were washed with 25% aq. ammonium chloride (2×10 ml).

The resulting THF solution was mixed with 1.33 gr of p-toluenesulfonic acid dissolved in 1 ml water at about 60° C. Then it was cooled to 25-30° C. 2 ml of water was added to the prepared solution. The mixture was cooled to 0-5° C. to obtain a yellow precipitation. The precipitated solid was filtered and dried to afford 1.0 gr of lapatinib ditosylate.

### Example 11

### Preparation of Lapatinib Ditosylate

1.0 gr “Intermediate-D” (compound of Formula D), 0.4 gr methylsulfonylethylamine hydrochloride, 10 ml methanol, 0.35 ml acetic acid and 1.1 ml N,N-diisopropylethylamine were mixed at 5-10° C. The resulting reaction mixture was stirred at 5-10° C. for an hour. Afterwards, 0.2 gr of sodium cyanoborohydride were added in two equal portions with 3 hours interval (2×0.1 gr). The resulting mixture was stirred at 5-10° C. for additional 16 hours. The reaction mixture is quenched with 3 ml of 25% NaOH aq. and 2 ml of water. The resulting mixture is stirred for 0.5 hours. The aqueous phase is separated and washed with 2.5 ml of THF. The combined THF extracts are washed with 25% aq. ammonium chloride (2×5 ml).

The resulting THF solution is mixed with 0.67 gr of p-toluenesulfonic acid dissolved in 1 ml water at about 60° C. Then it is cooled to 25-30° C. 1 ml of water is added to the prepared solution. The mixture is cooled to 0-5° C. to obtain yellow precipitation. The precipitated solid is filtered and dried to afford lapatinib ditosylate. Purity by HPLC: 91.76%

### Example 12

### Preparation of Crystalline Lapatinib Aldehyde Base

To a 1 L reactor, 50 g of lapatinib-aldehyde monotosylate and 300 ml of acetonitrile were added. To the resulting suspension a solution of 12.3 g sodium carbonate in 350 ml water was added. The resulting yellow suspension was stirred at 40° C. for 2 hours, and then cooled to room temperature (T(jacket)=25° C.) for an hour. The product was filtered in vacuum, washed with 50 ml of acetonitrile and dried for 16 hours in a vacuum oven at 40° C. Yield—30 gram (82%); purity—99.14%.

### Example 13

### Preparation of Crystalline Lapatinib Aldehyde Base

To a 3 L reactor 100 g of compound of formula C, 37.35 g of 5-formyl-2-furanboronic acid, 1.33 g of palladium acetate, 54.66 g of potassium carbonate, 750 ml of absolute ethanol and 750 ml of THF were added. The suspension was stirred and heated to reflux (T(jacket)=75° C.) for 40 minutes. The reaction mixture was cooled to room temperature (T(jacket)=20° C.) and diluted with 750 ml of THF and 750 ml of absolute ethanol. The resulting mixture was stirred at 25° C. for an hour. Inorganic salts were filtered off in vacuum, washed with 100 ml of absolute ethanol, 100 ml of THF and discarded. The filtrate combined with washings was transferred into a 10 L reactor equipped with a mechanical stirrer and a dropping funnel. 3 L of water was added dropwise into the solution of lapatinib-aldehyde base in EtOH/THF (1:1) for an hour (T(jacket)=20° C.). The resulting yellow suspension was stirred at RT (T(jacket)=20° C.) for 1.5 hour. The yellow solid was filtered in vacuum and washed over the filter with 100 ml of cold absolute ethanol. It was allowed to dry in a vacuum oven at 40° C. for 16 hours, and additional 24 hours in vacuum oven at 60° C. to give 92.56 g of final product (Yield—98.7%; Purity—99.12%)

### Example 14

### Preparation of Lapatinib Aldehyde Free Base

To a 100 ml round bottomed flask, 3.0 g of compound of Formula C, 1.0 g of 5-formyl-2-furanboronic acid, 0.013 g of palladium(II) acetate, 3.07 g of diisopropylethylamine, 22.5 ml of absolute ethanol and 22.5 ml of THF were added. The suspension was stirred and heated to reflux (T(jacket)=75° C.) for 90 minutes. The reaction mixture was filtered at hot stage to remove insoluble salts. The salts were washed with 3 ml THF and then with 3 ml of ethanol. The combined filtrate was cooled to room temperature (T(jacket)=20° C.) for 1.25 hours. The resulting yellow suspension was cooled to about 5° C. for about 0.5 hours and stirred at this temperature for an hour. The yellow solid was filtered in vacuum and washed over the filter with 3 ml of cold absolute ethanol. It was allowed to dry in a vacuum oven at 40° C. for 16 hours to give 2.1 g of lapatinib aldehyde free base (Yield—74.7%; Purity—99.5%).

### Example 15

### Preparation of Lapatinib Aldehyde

Compound of Formula C (3.0 kg), 5-formyl-2-furanboronic acid (0.913 kg), Palladium(II) Acetate (6.66 gr), ethanol 95% (33 L), N,N-Diisopropylethylamine (2.58 L), and THF (21 L) were mixed at 25° C. The solution was then heated to 65° C. and stirred for 5 hours.

The solution was filtered, and more THF was charged (3 L). The solution was cooled to 31° C. and part of the THF (14 L) from the reaction mixture was evaporated under vacuum (100-300 mm Hg) and a yellow precipitation was obtained. The mixture was stirred for half an hour and then evaporation of THF was completed.

Ethanol 95% (15 L) was added and the mixture was stirred for half an hour, then cooled to 10° C. for an hour and stirred at this temperature for 8 hours.

The mixture was then filtered and the obtained precipitate was washed twice with ethanol 95% (3 L). The wet solid was dried under vacuum (5-20 mmHg) at 35-45° C. to obtain 2.66 kg of dry lapatinib aldehyde. Purity by HPLC: 99.45%

### Example 16

### Preparation of Lapatinib Aldehyde

Compound of Formula C (3.5 kg), 5-formyl-2-furanboronic acid (1.09 kg), Palladium(II) Acetate (7.79 gr), ethanol 95% (38.5 L), N,N-diisopropylethylamine (3.02 L), and THF (24.6 L) were mixed at 25° C. The solution was then heated to 65° C. and stirred for 1.5 hours.

The solution was filtered, and more THF was charged (3.4 L). The solution was cooled to 18° C. and a yellow precipitation was obtained. The mixture was stirred for half an hour and then the THF of the reaction mixture was evaporated under vacuum (80-300 mm Hg) at 20-31° C. Ethanol 95% (17.5 L) was added, the mixture was stirred for half an hour, then cooled to 10° C. for an 5 hours and stirred at this temperature for 7 hours.

Then the mixture was filtered and the obtained precipitate was washed twice with ethanol 95% (3.5 L). The wet solid dried under vacuum (5-20 mmHg) at 25-38° C. to obtain 2.95 kg of dry lapatinib aldehyde. Purity by HPLC: 99.92%

### Example 17

### Preparation of Lapatinib Base

5.0 gr lapatinib aldehyde monotosylate, 2.0 gr methylsulfonylethylamine hydrochloride, 100 ml acetonitrile, 1.8 ml acetic acid and 2.5 gr sodium acetate were mixed at 25-30° C. The resulting reaction mixture was stirred at 25-30° C. for 1 hour. Afterwards, 4.1 gr of sodium triacetoxyborohydride were added. The resulting mixture was stirred at 25-30° C. for additional 1.5 hours. The reaction mixture was evaporated under reduced pressure to dryness.

The evaporation residue was dissolved in a mixture of 100 ml water, 20.0 gr sodium carbonate and 200 ml ethyl acetate resulting in a clear two-phase system. The organic phase was separated and, the obtained aqueous phase was extracted with 100 ml ethyl acetate. The separated organic phases were combined and washed with 100 ml water, dried over anhydrous sodium sulfate, filtered and evaporated to dryness. The evaporation residue was dissolved in 150 ml ethyl acetate and dried over anhydrous sodium sulfate and filtered.

65 ml of the dried filtrate in ethyl acetate were evaporated to dryness to get 1.6 gr of solid residue. The residue was re-crystallized from 13 ml ethyl acetate to form a suspension, which was filtered and dried to afford 0.88 gr of lapatinib base (Purity by HPLC—98.27%).

### Example 18

### Preparation of Lapatinib Base

5.0 gr lapatinib aldehyde monotosylate, 2.0 gr methylsulfonylethylamine hydrochloride, 150 ml ethyl acetate, 1.8 ml acetic acid and 2.5 gr sodium acetate were mixed at 25-30° C. The resulting reaction mixture was stirred at 25-30° C. for 1 hour. Afterwards, 4.1 gr of sodium triacetoxyborohydride were added. The resulting mixture was stirred at 25-30° C. for additional 1.5 hours.

100 ml water and 20 gr sodium carbonate were added to the reaction mixture resulting in a clear two-phase system. The organic phase was separated and the obtained aqueous phase was extracted again with 50 ml ethyl acetate. The separated organic phases were combined and washed with 100 ml water, dried over anhydrous sodium sulfate and filtered. The filtered sodium sulfate was washed with 50 ml ethyl acetate and the combined filtrate was evaporated to dryness to get 3.52 gr of sticky residue.

The residue was re-crystallized from 17 ml ethyl acetate to form a suspension, which was filtered and dried to afford 3.11 gr of lapatinib base (Purity by HPLC—94.49%).

### Example 19

### Preparation of Lapatinib Base

1.0 gr lapatinib aldehyde monotosylate, 0.4 gr methylsulfonylethylamine hydrochloride, 10 ml methyl acetate, 0.35 ml acetic acid and 0.5 gr sodium acetate were mixed at 25-30° C. The resulting reaction mixture was stirred at 25-30° C. for 1 hour. Afterwards, 0.82 gr of sodium triacetoxyborohydride were added. The resulting mixture was stirred at 25-30° C. for additional 2.5 hours.

The reaction mixture is neutralized by adding 4.0 gr sodium carbonate and 20 ml water. 20 ml methyl acetate is added to form a clear two-phase system. After organic phase separation the aqueous phase is extracted again with 10 ml methyl acetate. Combined organic phase is washed with 10 ml water, dried over anhydrous sodium sulfate, filtered and evaporated to dryness. The evaporation residue is re-crystallized from methyl acetate to form a suspension, which is filtered and dried to afford lapatinib base. Purity: 95.41 by HPLC.

### Example 20

### Preparation of Lapatinib Base

1.0 gr “Intermediate-D” monotosylate, 0.4 gr methylsulfonylethylamine hydrochloride, 20 ml dichloromethane, 0.35 ml acetic acid and 0.5 gr sodium acetate were mixed at 25-30° C. The resulting reaction mixture was stirred at 25-30° C. for 1 hour. Afterwards, 0.82 gr of sodium triacetoxyborohydride were added. The resulting mixture was stirred at 25-30° C. for additional 2.5 hours.

The reaction mixture is neutralized by adding sodium carbonate and water. 20 ml dichloromethane is added to form a clear two-phase system. After the organic phase is separated, the aqueous phase is extracted with 10 ml dichloromethane. The separated organic phases are combined and washed with 3×10 ml water, dried over anhydrous sodium sulfate, filtered, and evaporated to dryness. The evaporation residue is re-crystallized from dichloromethane to form a suspension, which is filtered and dried to afford lapatinib base.

### Example 21

### Preparation of Lapatinib Base

10.0 gr of lapatinib-aldehyde base, 5.5 gr of 2-methylsulfonyl-ethylamine HCl, 300 ml of ethyl acetate, 70 ml of DMF, 4.9 ml of acetic acid and 14.6 ml of N,N-diisopropylethylamine were mixed at 25° C. The reaction mixture was stirred at 35° C. for 1 hour and cooled to 25° C. Afterwards, 11.2 gr of sodium triacetoxyborohydride were added in one portion. The resulting mixture was stirred at 25° C. for 1.5 hours. The reaction mixture was quenched with 30 ml of 25% NH₄OH and with 70 ml of water and stirred at 25° C. for 0.5 hours. The resulting phases were separated. The organic phase was washed with 25% NH₄Cl aqueous solution (2×50 ml) and with 200 ml of water.

To 148 ml of organic phase, 0.11 gr of tetra-butylammonium hydrogen sulfate was added. The mixture was stirred at 25° C. for 30 min. Then, it was concentrated in vacuum to about 50 ml. The resulting off-white suspension was stirred at 25° C. in a 100 ml round-bottomed flask for 16 hours. The solid was filtered off in vacuum and washed with 10 ml of fresh ethyl acetate. The solid was dried in a vacuum oven for 16 hours at 50° C. 3.18 gr (yield 51.9%). Purity: 99.72%.

### Example 22

### Preparation of Lapatinib Base

15.0 gr of lapatinib-aldehyde base, 8.25 gr of 2-methylsulfonyl-ethylamine HCl, 450 ml of ethyl acetate, 105 ml of DMF, 7.35 ml of acetic acid and 21.9 ml of N,N-diisopropylethylamine were mixed at 25° C. The reaction mixture was stirred at 35° C. for 1 hour and then cooled to 25° C. Afterwards, 29.44 gr of sodium triacetoxyborohydride were added by one portion. The resulting mixture was stirred at 25° C. for 1.5 hours. The reaction mixture was then quenched with 135 ml of water and 16.5 ml of 47% NaOH aqueous and stirred at 25° C. for 0.5 hours. The aqueous and organic phases were separated.

To 175 ml of organic phase 25 ml of THF were added. The resulting solution was washed with a mixture of 5.5 ml of 47% of NaOH and 300 ml of water and the phases were separated. 2.5 ml of acetic acid were added to the organic phase. The mixture was stirred at 25° C. for 15 minutes and transferred into a separating funnel where the organic layer was isolated and washed with 100 ml of water. The organic phase was concentrated in vacuum to about 50 ml. The resulting off-white suspension was stirred at 25° C. in a 100 ml round-bottomed flask for 16 hours. The solid was filtered off in vacuum and washed over the filter with 15 ml of fresh ethyl acetate. The solid was dried in a vacuum oven for 16 hours at 50° C. 3.76 gr (yield 48.9%). Purity: 99.61%.

### Example 23

### Preparation of Lapatinib Ditosylate

100.0 gr (0.211 mol) of lapatinib aldehyde and 54.6 gr (0.342 mol) of methylsulfonylethylamine HCl were mixed with 500 ml THF and 100 ml DMF. 48 ml acetic acid and 147 ml diisopropylethylamine were added. The reaction mixture was heated to 35° C. and stirred at this temperature for 1 hour and cooled to 20-25° C. 111.8 gr (0.528 mol) of sodium triacetoxyborohydride were added and stirred for additional 2 hours to complete the reaction.

1500 ml ethyl acetate and 300 ml water were added to the reaction mixture. Then 110 ml of 47% sodium hydroxide solution was added. The mixture allowed for the phase separation. The organic phase was washed twice with 500 ml of 25% aqueous ammonium chloride solution. The resulting organic phase was washed twice with 300 ml of water. The resulting organic solution was concentrated to dryness. 350 ml of DMF is added to the evaporation residue to form lapatinib solution in DMF.

The resulting organic solution was heated to about 40° C. and filtered to remove foreign particles. 263 ml DMF were added to the filtrate and heated again to about 40° C. 80.3 g (0.442 mol) of p-toluenesulfonic acid were added to the lapatinib solution in DMF. The solution was seeded with pure lapatinib ditosylate and cooled slowly from 40° C. to about 0° C. for 6 hours. The formed slurry was stirred at about 0° C. for 9 hours, cooled additionally to about (−10)° C. to complete precipitation and stirred at this temperature during 2 hours. The crude product was filtered and washed with 60 ml of cold DMF to get 248.2 gr of wet lapatinib ditosylate crude.

244.8 gr of the wet lapatinib ditosylate crude were slurried in 490 ml of DMF at about 40° C. during 2 hours, cooled to about (−10)° C. during 2 hours and stirred at this temperature during additional 17 hours. The precipitated product was filtered, washed with 60 ml DMF and dried at 70° C. under reduced pressure during overnight to give 133.7 gr (72% yield) of the lapatinib ditosylate purity: 99.73%.

### Example 24

### Preparation of Lapatinib Ditosylate

P-toluenesulfonic acid was added to a solution of lapatinib-base in 5V dimethylformamide, 8.03 gr (2 eq) to obtain a brownish solution. The solution was seeded with lapatinib ditosylate at 40° C., then it was stirred for 1 hour, to obtain a yellow suspension. Then, it was cooled to 0° C. for 6 hours, and stirred for 10 hours. The resulting suspension was deep-cooled to −10° C. for 2 hours, and stirred for 2 hours. The precipitate was filtered to obtain lapatinib ditosylate.

What is claimed is:

1. Lapatinib salt having purity levels of more than about 99.7% weight by HPLC.

2. The lapatinib salt of claim 1, having purity levels of more than about 99.8% weight by HPLC.

3. The lapatinib salt of claim 1, having purity levels of more than about 99.9% weight by HPLC.

4. A composition of 3-chloro-4-(3-fluorobenzyloxy)aniline of the following Formula B: 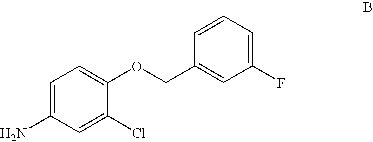
having less than 0.3% area by HPLC of any one of 3-chloro-4-(benzyloxy)aniline, 4-(3-fluorobenzyloxy)aniline, 4-benzyloxyaniline, 3-chloro-4-hydroxyaniline, 4-aminophenol or a combination thereof.

5. A composition of N-[3-chloro-4-(3-fluorobenzyloxy)-phenyl]-6-iodoquinazolin-4-amine of the following Formula C: 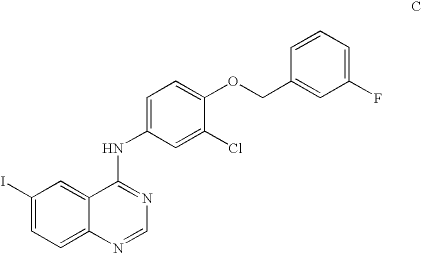
having less than 0.3% area by HPLC of any one of [3-chloro-4-(benzyloxy)-phenyl]-(6-iodoquinazolin-4-yl)-amine, [4-(3-fluorobenzyloxy)-phenyl]-(6-iodoquinazolin-4-yl)-amine, 4-benzyloxyphenyl-(6-iodoquinazolin-4-yl)-amine, 3-chloro-4-hydroxyphenyl-(6-iodoquinazolin-4-yl)-amine, 4-hydroxyphenyl-(6-iodoquinazolin-4-yl)-amine, or a combination thereof.

6. A process for preparing the compound of Formula C comprising:
reducing a first reaction mixture of a compound of Formula A using an iron/ammonium chloride system in the presence of a first organic solvent of a C₁-C₄ alcohol and water;
removing iron oxide from the reaction mixture;
removing inorganic salt;
adding 4-chloro-6-iodoquinazoline and a second organic solvent to obtain a second reaction mixture; and
heating the second reaction mixture to obtain the compound of Formula C.

7. The process of claim 6, wherein the first reaction mixture is heated to about reflux temperature for about 2 to about 30 hours.

8. The process of claim 6, wherein the first reaction mixture is heated to about reflux temperature for about 6 to about 12 hours.

9. The process of claim 6, wherein the second organic solvent is selected from the group consisting of acetonitrile, dimethylsulfoxide, and C₁-C₄ alcohols.

10. The process of claim 6, wherein after the addition of the second organic solvent, the reaction mixture is heated to a temperature of about reflux.

11. The process of claim 6, wherein the inorganic salt is removed by extraction with a mixture of water and dichloromethane.

12. A process for purifying the compound of Formula C, comprising recovering the compound of Formula C from a mixture of compound C in a C₁-C₄ alcohol.

13. The process of claim 12, wherein the amount of the C₁-C₄ alcohol is about 10V to about 40V.

14. The process of claim 12, wherein the C₁-C₄ alcohol is methanol.

15. The process of claim 6 further comprising converting the compound of Formula C to lapatinib ditosylate, wherein the obtained lapatinib ditosylate has less than 0.3% area by HPLC of any one of [3-chloro-4-(benzyloxy)-phenyl]-(6-iodoquinazolin-4-yl)-amine, [4-(3-fluorobenzyloxy)-phenyl]-(6-iodoquinazolin-4-yl)-amine, 4-benzyloxyphenyl-(6-iodoquinazolin-4-yl)-amine, 3-chloro-4-hydroxyphenyl-(6-iodoquinazolin-4-yl)-amine, 4-hydroxyphenyl-(6-iodoquinazolin-4-yl)-amine, or a combination thereof.

16. A composition of lapatinib aldehyde monotosylate of the following Formula D: 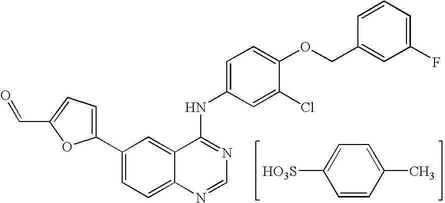
having less than 0.3% area by HPLC of any one of 5-[4-[3-chloro-4-(benzyloxy)-phenylamino]-quinazolin-6-yl]-furan-2-carbaldehyde, 5-[4-[4-(3-fluorobenzyloxy)-phenylamino]-quinazolin-6-yl]-furan-2-carbaldehyde, 5-[4-(4-benzyloxyphenylamino)-quinazolin-6-yl]-furan-2-carbaldehyde, 5-[4-(3-chloro-4-hydroxyphenylamino)-quinazolin-6-yl]-furan-2-carbaldehyde, 5-[4-(4-hydroxyphenylamino)-quinazolin-6-yl]-furan-2-carbaldehyde, or a combination thereof.

17. Isolated lapatinib aldehyde base of the following Formula D1:

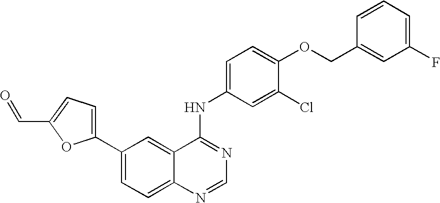

18. The lapatinib aldehyde base of claim 14, wherein the lapatinib aldehyde base is solid.

19. The lapatinib aldehyde base of claim 17, having less than 0.3% area by HPLC of any one of 5-[4-[3-chloro-4-(benzyloxy)-phenylamino]-quinazolin-6-yl]-furan-2-carbaldehyde, 5-[4-[4-(3-fluorobenzyloxy)-phenylamino]-quinazolin-6-yl]-furan-2-carbaldehyde, 5-[4-(4-benzyloxyphenylamino)-quinazolin-6-yl]-furan-2-carbaldehyde, 5-[4-(3-chloro-4-hydroxyphenylamino)-quinazolin-6-yl]-furan-2-carbaldehyde, 5-[4-(4-hydroxyphenylamino)-quinazolin-6-yl]-furan-2-carbaldehyde, or a combination thereof.

20. A process for preparing the compound of Formula D1 comprising: heating a reaction mixture of a compound of Formula C, 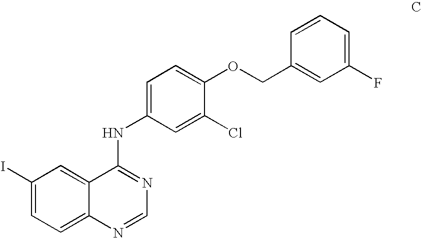
5-formyl-2-furanboronic acid, a palladium catalyst, a base, and a polar organic solvent to about 40° C. to about 110° C.;
removing salts from the reaction mixture; and
recovering lapatinib aldehyde base as a precipitate from the reaction mixture; and
wherein the palladium catalyst is selected from the group consisting of palladium(II) acetate, palladium(II) chloride, and palladium tetrakistriphenylphosphine.

21. The process of claim 20, wherein the recovered lapatinib aldehyde base is further combined with p-toluenesulfonic acid to obtain a compound of Formula D.

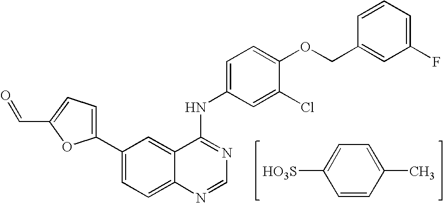

22. The process of claim 20, wherein the palladium catalyst is palladium(II) acetate.

23. The process of claim 20, wherein the base is selected from the group consisting of alkali carbonates, alkali bicarbonates, alkali acetates, alkali phosphates, alkali hydroxide, aliphatic tertiary amines or secondary amines having alkyl groups independently selected from the group consisting of C₁-C₄ alkyls, C₂-C₁₀ cyclic or polycyclic tertiary amines or secondary amines, and tertiary amines or secondary amines combined from any C₁-C₄ aliphatic or aromatic or heterocylic group.

24. The process of claim 23, wherein the base is diisopropylethylamine.

25. The process of claim 20, wherein the polar organic solvent is selected from the group consisting of tetrahydrofuran, dioxane, C₁-C₄ alcohols, C₂-C₁₀ glycols, glycerol, and mixtures thereof.

26. The process of claim 25, wherein the polar organic solvent is a mixture of ethanol and tetrahydrofuran.

27. A process for purifying lapatinib aldehyde monotosylate, comprising triturating lapatinib aldehyde monotosylate with an organic solvent selected from the group consisting of C₁-C₄ alcohols, acetone, acetonitrile, and tetrahydrofuran.

28. A process of preparing lapatinib ditosylate, comprising converting the composition of claim 4 or 5 or the isolated lapatinib aldehyde base of claim 17 to lapatinib ditosylate.

29. A process for preparing lapatinib base, comprising:
providing a first reaction mixture containing lapatinib aldehyde base or its salt, methylsulfonylethylamine or its hydrochloride salt, acetic acid, an inorganic base, and an organic solvent;
providing a second reaction mixture by adding a reducing agent to the first reaction mixture;
adding water and separating organic phase from aqueous phase; and recovering precipitated lapatinib base from the organic phase.

30. The process of claim 29, wherein the inorganic base is selected from the group consisting of alkali carbonates, alkali bicarbonates, alkali phosphates, and alkali acetates.

31. The process of claim 29, wherein the inorganic base is sodium acetate or potassium acetate.

32. The process of claim 29, wherein the reducing agent is sodium triacetoxyborohydride.

33. The process of claim 29, wherein the organic solvent is selected from the group consisting of dimethylformamide, dimethylacetamide, dichloromethane, dimethyl carbonate, diethyl carbonate, toluene, acetonitrile, tetrahydrofuran, and C₁-C₆ esters;
wherein, if toluene, acetonitrile, or tetrahydrofuran is selected, then the second reaction mixture is evaporated to dryness before the step of producing a biphasic mixture, and wherein a biphasic mixture is produced by adding a mixture of water and ethyl acetate or a mixture of water and isobutyl acetate.

34. The process of claim 33, wherein the organic solvent is a C₁-C₄ ester.

35. The processes of claim 33, wherein the organic solvent is selected from the group consisting of ethyl acetate, dimethylformamide, and dimethylacetamide.

36. The process of claim 29, wherein the first reaction mixture is maintained at about room temperature from about 15 minutes to about 24 hours.

37. The process of claim 29, wherein an inorganic base is added to the second reaction mixture.

38. A process for preparing lapatinib base comprising:
providing a first reaction mixture containing compound D or its salt, methylsulfonylethylamine or its hydrochloride salt, acetic acid, N,N-diisopropylethylamine, ethyl acetate, and/or tetrahydrofuran, and a water miscible organic solvent selected from dimethylformamide and dimethylacetamide; 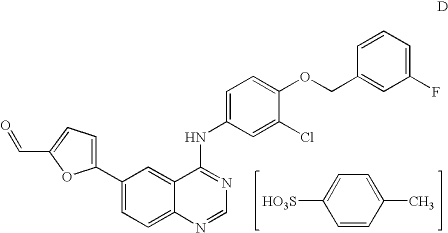
providing a second reaction mixture by adding a reducing agent to the first reaction mixture;
adding water, and separating organic phase from aqueous phase; and
isolating lapatinib base from the organic phase.

39. The process of claim 38, wherein the reducing agent is sodium triacetoxyborohydride.

40. The process of claim 38, wherein the first reaction mixture is maintained at about room temperature for about 15 minutes to about 24 hours.

41. The process of claim 38, wherein an inorganic base is added to the second reaction mixture.

42. A process for purifying lapatinib base comprising providing a suspension or a solution of lapatinib base and an organic solvent selected from the group consisting of C₃-C₇ ketones; and collecting a precipitate.

43. The process of claim 42, wherein the organic solvent is ethyl acetate.

44. The process of claim 42, wherein the amount of the solvent is about 4V to about 30V.

45. The process of claim 42, wherein the suspension is maintained at about 0° C. to about room temperature.

46. A process for preparing lapatinib ditosylate, comprising:
obtaining lapatinib base by combining the compound of Formula D, or the compound of Formula D1, and methylsulfonylethylamine free base or its salt, an organic solvent, an organic base, acetic acid, and a reducing agent; 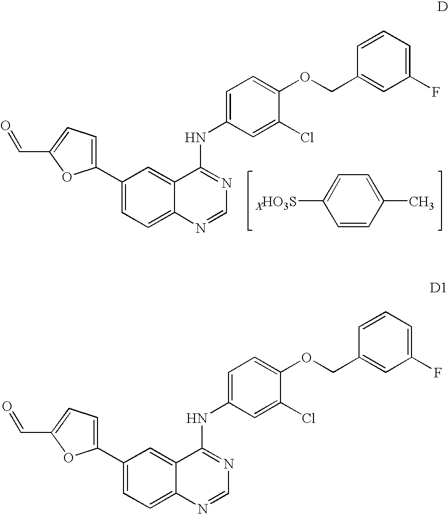
forming a mixture of about 15V to about 30V of ethyl acetate, about 3V to about 7V of tetrahydrofuran, and about 1V to about 10V of dimethylformamide or dimethylacetamide, and water; separating organic phase from aqueous phase; and adding p-toluenesulfonic acid to the organic phase to obtain lapatinib ditosylate.

47. The process of claim 46, wherein the organic solvent is selected from the group consisting of C₁-C₄ alcohols, acetic acid, ethyl acetate, dichloromethane, N,N-dimethylformamide, N,N-dimethylacetamide, acetonitrile, tetrahydrofuran, dimethoxyethane, methyl tert butyl ether, toluene, and mixtures thereof.

48. The process of claim 46, wherein the organic base is a trialkylamine having alkyl groups selected from the group consisting of C₁-C₄ alkyls.

49. The process of claim 46, wherein the reducing agent is selected from the group consisting of sodium borohydride, and sodium cyanoborohydride.

50. The process of claim 46, wherein the obtained lapatinib base is extracted into a mixture of about 15V to about 30V of ethyl acetate, about 3V to about 7V of tetrahydrofuran, and about 1V to about 10V of dimethylformamide.

51. The process of claim 46, wherein the reaction mixture is maintained at a temperature of about 0° C. to about 30° C. for about an hour.

52. The process of claim 46, wherein the reducing agent is added dropwise within about 30 minutes.

53. A process for the purification of lapatinib ditosylate, comprising triturating lapatinib ditosylate from methanol.

54. A process for preparing lapatinib ditosylate, comprising the following steps:
a) reducing a compound of Formula A 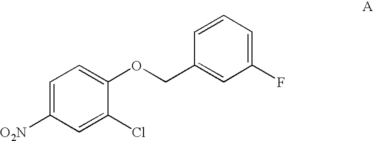
using an iron/ammonium chloride system in the presence of a C₁-C₄ alcohol, and water; removing iron oxide from the reaction mixture; removing inorganic salt from the reaction mixture; adding 4-chloro-6-iodoquinazoline and a second organic solvent to obtain a second reaction mixture; and heating the second reaction mixture to obtain a compound of Formula C; 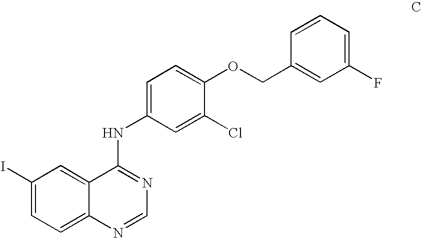
b) heating a reaction mixture of the compound of Formula C, 5-formyl-2-furanboronic acid, a palladium catalyst, a base, and a polar organic solvent; removing salts from the reaction mixture; and recovering lapatinib aldehyde base as a precipitate from the reaction mixture; and optionally adding p-toluenesulfonic acid; and
c) combining lapatinib aldehyde monotosylate, or lapatinib aldehyde base, and methylsulfonylethylamine free base or its salt, in the presence of a third organic solvent, an organic base, acetic acid, and a reducing agent to obtain lapatinib base; forming a mixture of about 15V to about 30V of ethyl acetate, about 3V to about 7V of tetrahydrofuran, and about 1V to about 10V of dimethylformamide or dimethylacetamide, and water; separating organic phase from aqueous phase; and adding p-toluenesulfonic acid to the organic phase to obtain lapatinib ditosylate;
wherein removing the salt from the reaction mixture in step a) is achieved by extraction with a mixture of water and dichloromethane; and wherein the palladium catalyst in step b) is selected from the group consisting of palladium(II) acetate, palladium(II) chloride, and palladium tetrakistriphenylphosphine.

55. The process of claim 54, wherein the polar organic solvent is a mixture of ethanol and tetrahydrofuran.
